(19) United States
(12) Patent Application Publication
(10) Pub. No.: US 2007/0020301 A1
(43) Pub. Date: Jan. 25, 2007

(54) Beautification method

(75) Inventors: Kazunori Shimagami, Tokyo (JP); Rumiko Ataku, Kanagawa (JP)

Correspondence Address: KNOBBE MARTENS OLSON & BEAR LLP, 2040 MAIN STREET, FOURTEENTH FLOOR, IRVINE, CA, 92614, US

(73) Assignee: KAO CORPORATION, Chuo-Ku Tokyo (JP)

(21) Appl. No.: 10/569,855

(22) PCT Filed: Aug. 27, 2004
(86) PCT No.: PCT/JP04/12804

§ 371 Date: Oct. 6, 2006

(30) Foreign Application Priority Data
Aug. 29, 2003 (JP) 2003-306503

(51) Int. Cl. A61K 8/02; A61B 19/00

(52) U.S. Cl. 424/401; 128/898

(57) ABSTRACT

It is intended to provide a cosmetic method that comprises carrying out a beauty treatment in exact timing with an increase in the concentration of a medicinal component that has been taken in blood so as to establish a favorable cosmetic effect owing to synergy of both. A subject person is made to take a medicinal component and then at least one of a bathing treatment, a beauty treatment and a napping treatment is carried out.

### TECHNICAL FIELD

This invention relates to a cosmetic method that is to be carried out on a client, etc. by an esthetic worker, etc.

### BACKGROUND ART

Various massage treatments for cosmetic purposes have been practiced in esthetic salons, esthetic corners in cosmetic stores and the like. As the most common example thereof, facial massage for a facial beauty treatment may be cited.

In addition to mere facial massage, there have been recently proposed a technique of intensively massaging around eyes (see, for example, JP-A-2001-37542) and a technique of relaxing body and mind by combining a facial massage with a special massage treatment on body areas other than the face (see, for example, JP-A-2002-282324).

In recent years, however, there has been proposed that cosmetic effects are improved not merely by skin massaging but by keeping in a mentally relaxed state or enhancing cosmetic effects in the body with the use of supplements or the like. That is to say, it has been strongly required to develop a cosmetic method of establishing excellent cosmetic effects that is different from the existing cosmetic methods based on physical massaging.

### DISCLOSURE OF THE INVENTION

An object of the present invention, which has been achieved under the above-described circumstances, is to provide a cosmetic method by which highly excellent cosmetic effects can be established by combining the intake of medicinal components and a beauty treatment and so on.

To achieve the above-described object, the first gist of the present invention resides in a cosmetic method which comprises making a subject person to take a medicinal component and then carrying out at least one of a bathing treatment, a beauty treatment and a napping treatment. The second gist thereof resides in, among all, a cosmetic method which comprises making a subject person to take a medicinal component and carrying out a bathing treatment and then a beauty treatment.

The third gist of the present invention resides in a cosmetic method which comprises making a subject person to take a first medicinal component, then carrying out a bathing treatment, making him/her to take a second medicinal component and then carrying out a beauty treatment. The fourth gist thereof resides in, among all, a cosmetic method which comprises making a subject person to take a first medicinal component, then carrying out a bathing treatment, making him/her to take a second medicinal component, then carrying out a beauty treatment, then carrying out a napping treatment and making him/her to take a third medicinal component.

Moreover, the fifth gist of the present invention resides in a cosmetic method which comprises carrying out a cosmetic method according to any of the first to fourth gists as described above and then making a subject person to carry out a recovery care comprising a skin rest treatment combined with a skin recovery treatment in the home, etc.

The term “beauty treatment” as used in the present invention means a treatment for cosmetic purpose that is performed on a subject person by an esthetic worker or the like, while the term “recovery treatment” means a treatment for cosmetic purpose that is performed by a subject person himself/herself, who has already had treatment(s) such as the above-described beauty treatment, in the home, etc.

The term “skin rest” means a state wherein skin stimulation is minimized and minimum skin care is carried out.

In the cosmetic method according to the present invention, a subject person is made to take a medicinal component and then at least one of a bathing treatment, a beauty treatment and a napping treatment is carried out. Thus, the medicinal component is taken into tissues in the body in the state where the blood circulation is promoted by the above-described treatment and the body and mind of the subject person have been both relaxed. As a result, the medicinal component is taken at a high speed and at a high concentration and thus extremely favorable cosmetic effects can be established, compared with the case in which the medicinal component is merely taken.

In the case where a subject person is made to take the medicinal component and the bathing treatment and then the beauty treatment are carried out in the above-described cosmetic method, the absorption of the medicinal component is further promoted and still improved cosmetic effects can be obtained.

In the case of making the subject to take not merely a first medicinal component, i.e., a method comprising making a subject person to take a first medicinal component, then carrying out a bathing treatment, making him/her to take a second medicinal component and then carrying out a beauty treatment, more improved cosmetic effects can be obtained owing to synergy of the above-described 2 medicinal components.

For the same reason, still improved cosmetic effects can be obtained owing to synergy of 3 medicinal components by making a subject person to take a first medicinal component, then carrying out a bathing treatment, making him/her to take a second medicinal component, then carrying out a beauty treatment, then carrying out a napping treatment and making him/her to take a third medicinal component.

Moreover, the cosmetic effects can be further improved by carrying out such a cosmetic method as described above and then making a subject person to carry out a specific recovery care by himself/herself in the home, etc.

### BRIEF DESCRIPTION OF THE DRAWINGS

FIG. 1

FIG. 1 illustrates the constitution of a cosmetic course in an embodiment of the present invention.

FIG. 2

FIG. 2 illustrates the constitution of a cosmetic course in an embodiment of the present invention.

FIG. 3

FIG. 3 illustrates the constitution of a cosmetic course in an embodiment of the present invention.

FIG. 4

FIG. 4 is a diagram showing blood circulation conditions in the skin, and changes in the concentration of a medicinal component in blood and the concentration of growth hormone in blood with-the passage of time.

### BEST MODE FOR CARRYING OUT THE INVENTION

Now, best modes for carrying out the present invention will be illustrated.

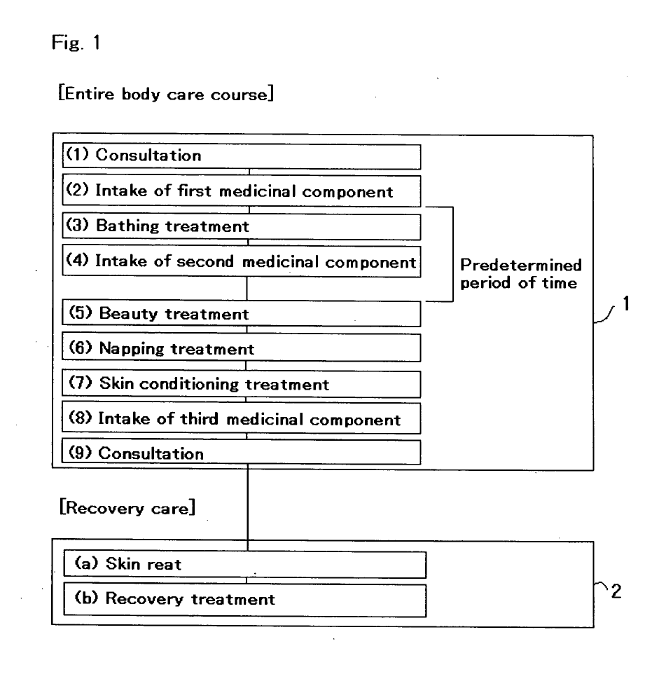

The method according to the present invention, which is a cosmetic method to be carried out on a client by an esthetic worker, etc., includes, for example, 3 courses as shown in FIG. 1 to FIG. 3. A suitable course may be selected in response to the client's request or following the esthetic worker's advice.

In FIG. 1, the course in the frame 1 is named “entire body care course”. This course, which is performed by an esthetic worker (hereinafter referred to as “esthetician”) on a client (hereinafter referred to as “subject person”) in an esthetic salon or the like, comprises the following 9 steps: (1) consultation; (2) intake of a first medicinal component; (3) a bathing treatment; (4) intake of a second medicinal component; (5) a beauty treatment; (6) a napping treatment; (7) a skin conditioning treatment; (8) intake of a third medicinal component; and (9) consultation.

In the above-described step “(1) consultation”, an esthetician questions a subject person to understand his/her mental, skin and physical balances. In this step, it is important to understood contraindications in performing the treatments such as the physical conditions, clinical history, whether or not being pregnant or allergic and soon. If necessary, physical data such as blood pressure, body fat percentage, height and weight and skin data such as skin moisture content, sebum content, moisture content in corneous layer and peeling pattern of corneous layer are measured by using instruments. Based on the results of the questions and measurement data, the most suitable treatment plan for the subject person is conceived.

In the following step “(2) intake of a first medicinal component”, a supplement containing a medicinal component suitable for the subject person's conditions is prepared based on the treatment plan as described above and orally administered to the subject person. As the above-described supplement, those listed in the following Table 1 are prepared. In this step, it is usually appropriate to take a supplement containing components having slimming effects (effects of promoting perspiration and fat burning) in the form of a hot drink. From a cosmetic viewpoint, it is appropriate that any preparation contains vitamin C and vitamin B₁₂. Examples of the preparation include a tea-like drink prepared by mixing raspberry ketone, dietary fiber, capsaicin and ginger extract at an appropriate ratio and decocting the mixture.

TABLE 1

| | | Natural | | |
|---|---|---|---|---|
| | Vitamin | component | Mineral | Others *1 |
| Allergy | beta- | Catechin | | α-Linolenic |
| | Carotene | Perilla | | acid |
| | Vitamin B₆ | Chinese | | β-Linolenic |
| | Vitamin B₁₂ | blackberry | | acid |
| | Vitamin C | extract | | |
| | | Propolis | | |
| Anti- | beta- | Salacia | Calcium | Konjak |
| obesity | Carotene | Citrus | Iron | mannan |
| diet | Vitamin B | Gymnema | Sodium | Marine |
| | family | Raspberry | Phosphorus | algae |
| | Niacin | ketone | Copper | |
| | Vitamin B₁₂ | Mulberry | Zinc | |
| | Vitamin C | leaf | Manganese | |
| | | Capsaicin | Iodine | |
| | | Ginger | | |
| | | extract | | |
| Consti- | Vitamin B | Lacto- | | Marine |
| pation | family | bacillus | | algae |
| | Vitamin B₁₂ | bifidus | | Konjak |
| | Vitamin C | Oligosac- | | mannan |
| | | charide | | Burdock |
| Energy- | beta- | Glucose | | Amino acids |
| supply | Carotene | Oligosac- | | |
| | Vitamin B | charide | | |
| | family | | | |
| | Vitamin B₁₂ | | | |
| | Vitamin C | | | |
| | Vitamin E | | | |
| | Niacin | | | |
| Menopausal | Vitamin K | Isoflavone | Calcium | |
| disorder | Vitamin D | Glucosamine | Phosphorus | |
| Bone care | Vitamin B | Chondroitin | Magnesium | |
| | family | St. John's | | |
| | Vitamin B₁₂ | word | | |
| | Vitamin C | | | |
| Hyper- | Vitamin B₁₂ | Ginkgo leaf | Iron | |
| sensitivity | Vitamin C | Ginger | | |
| to cold | | Korean | | |
| Swelling | | ginseng | | |
| Stiff neck | | Red pepper | | |
| Hair care | Vitamin B₂ | Isoflavone | Zinc | |
| | Vitamin B₆ | | Iodine | |
| | Vitamin B₁₂ | | | |
| | Vitamin C | | | |
| | Biotin | | | |
| | Pantothenic | | | |
| | acid | | | |
| Skin care | beta- | Job's tears | Manganese | α-Linolenic |
| | Carotene | Turmeric | Zinc | acid |
| | Vitamin B | | Calcium | β-Linolenic |
| | family | | | acid |
| | Vitamin B₁₂ | | | Oleic acid |
| | Vitamin C | | | Linolic acid |
| | Vitamin E | | | Amino acids |
| | Niacin | | | Proteins |
| | | | | Peptides |

*1: Unsaturated fatty acids, dietary fiber, etc.

In the following step “(3) bathing treatment”, the subject person is bathed. This bathing treatment makes the subject person relax and freshen up in body and mind. Owing to perspiration, body wastes are eliminated and thus the body can be cleaned up not only in pores and skin surface but also from the inside. Furthermore, the blood circulation is promoted and thus the tissue metabolism can be also promoted. Lactic acid is eliminated form the body, which contributes to the recovery from muscular fatigue. The term “bathing” as used in the present invention involves not only so-called “bathing” (i.e., soaking in a bathtub) but also just showering, taking a sauna, having a steamer bath, etc.

In the case of soaking a bathtub, it is still favorable from a cosmetic viewpoint to add an appropriate bath agent to the bathwater to thereby further improve the bating effects. In the case of using a hydrogencarbonic acid salt (NaHCO₃), for example, the above-described hydrogencarbonic acid salt induces saponification of sebum and thus skin pores can be unstuffed. In the case of using a bath agent containing metasilicic acid, skin turnover can be promoted and thus roughed skin can be improved, thereby smoothening the skin. It is also possible to employ bath agents of different types according to seasons by taking environmental effects on the skin into consideration. For example, use may be appropriately made of a bath agent having an antioxidant effect in spring, a bath agent having a pore-care effect in early summer, a bath agent having a whitening effect in summer, a bath agent having a corneous layer-care effect in fall, a bath agent having a moisturizing effect in early winter and a bath agent having a metabolism promoting effect in winter.

The time required for the bathing treatment varies depending on the contents of the treatment. In usual, it is suitable to carry out the treatment for about 10 to 20 minutes while considering the digestion and absorption time of the first supplement taken before the bathing and the time of starting the subsequent beauty treatment. Within such a treatment time, the above-described bathing effects can be fully achieved while giving little burden to the body.

In the following step “(4) intake of a second medicinal component”, a second supplement containing a medicinal component suitable for the subject person's conditions is prepared based on the treatment plan as described above and orally administered to the subject person. As the second supplement as described above, appropriate components are selected from those listed in the above Table 1 and blended. In this step, it is usually appropriate to take a supplement in the form of a concentrated drink that contains a component having skin care effect. As an example of the formulation, a concentrated drink containing a mixture of vitamin C, vitamin B₁₂, vitamin E, vitamin B family, beta-carotene and amino acids at an appropriate ratio may be cited.

In the following step “(5) beauty treatment”, the following operations are performed on the subject person. Namely, the subject person has hand massages on the dorsal, the backside of lower limbs and the head with the use of a massage agent. The face and the part from the neck to the breast (the decollete) are subjected to a series of operations including: cleansing→ hand massaging with the use of a massage agent→ impartment of an active ingredient by masking→ impartment of a cosmetic lotion, an emulsion and a cosmetic essence suitable for the skin conditions of the subject person using a mask. The subject person further has hand massages with the use of a massage agent on the abdomen and the front side of lower limbs. However, these operations for respective parts may be performed in an optional order.

It is generally appropriate that the above-described hand massages are performed in 3 steps including physically stimulating operations for promoting blood circulation such as rubbing, kneading and tapping, balancing operations such as pushing reflex points and finger pressure, and operations for maintaining improved blood circulation such as stroking the skin and heating. It is also possible to appropriately combine these operations with blood circulation-promoting operations such as wrapping and packing.

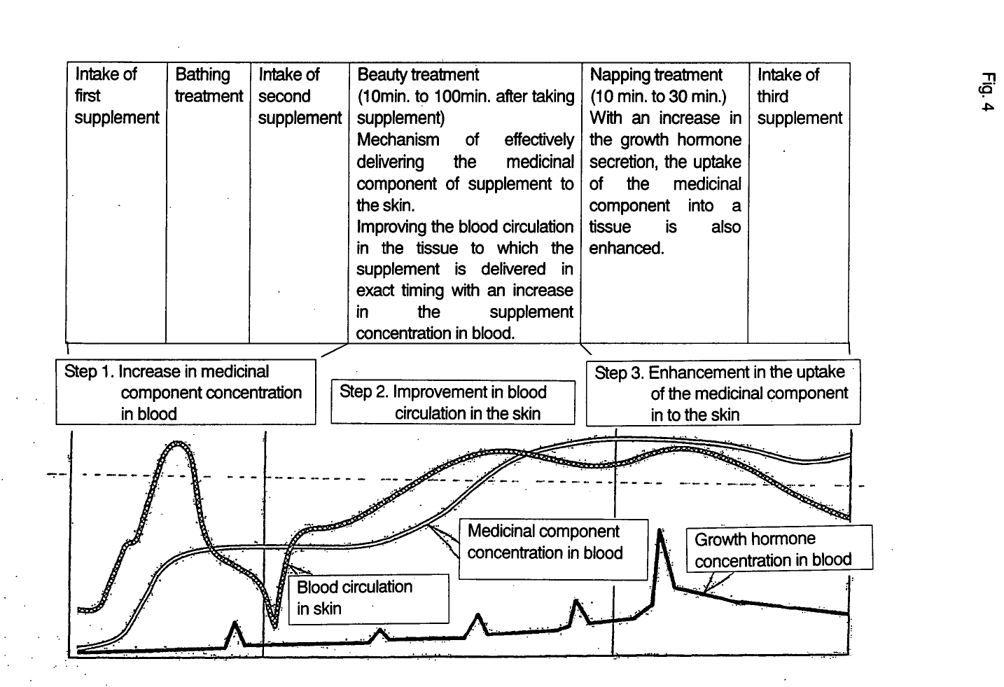

It is appropriate to start the above-described step “(5) beauty treatment” after a definite time has passed since the intake of the first medicinal component as described above and thus the concentration of the medicinal component in blood has been elevated to a certain level. By starting the beauty treatment after the concentration of the first medicinal component in blood has been elevated to a certain level and then maintained as FIG. 4 shows, the blood circulation in the skin can be promoted and the concentration can be further elevated. Since the time required for the elevation of the above-described medicinal component in blood varies depending on various factors such as the type of the medicinal component, the subject person's age, the form of the taken supplement (aqueous solution, a tablet, a capsule, etc.), intake time (day or night) and the condition of the subject person's stomach (empty or full), the time from the bathing treatment to the initiation of the beauty treatment should be appropriately controlled so as to perform the beauty treatment at the perfect timing. The above-described time is usually set to about 10 to 100 minutes after the intake of the first medicinal component.

In the following step “(6) napping treatment”, the subject person is allowed to have a nap. Owing to the napping, the secretion of growth hormone can be promoted and the medicinal component, the concentration of which has been elevated by the previous beauty treatment, can be more efficiently utilized in cells and tissues (see FIG. 4). Among all, the improved utilization of amino acids can appropriately promote tissue recovery. By having a nap while warming the body, the blood circulation and metabolism having been activated by the previous beauty treatment are gently cooled down and the effects of the beauty treatment can be sustained over a longer time. Since the contact stimulations given in the previous beauty treatment still continue while the subject person is left as such during the nap, considerable relaxing effects can be achieved by the gap between these treatments. In this step, it is also possible to perform, for example, masking and body wrapping treatments.

Although the napping time in the above-described napping treatment can be optionally determined, it is appropriately set to 10 to 30 minutes in usual. In the case where the napping time is shorter than 10 minutes, the above-described effects of napping can be hardly achieved in practice. In the case where the napping time exceeds 30minutes, on the other hand, the subject person would feel fatigue or be kept under unnecessary restraint.

In the following step “(7) skin conditioning treatment”, a skin conditioning treatment is carried out on the subject person. In this treatment, the skin of the subject person is conditioned appropriately by using a cosmetic lotion, an emulsion, a full body care cream, an essence suitable for the subject person's skin conditions, a finishing cream and so on in usual.

In the following step “(8) intake of a third medicinal component”, a supplement containing a medicinal component suitable for the subject person's conditions is prepared based on the treatment plan as described above and orally administered to the subject person. The above-described supplement may be appropriately selected from those listed in the above Table 1 and blended. In this step, it is usually appropriate to select a supplement having an awakening effect and an energy-supplying effect. As an example of the formulation, a candy containing a mixture of glucose, niacin vitamins such as niacin, amino acids, hydrocarbonates (monosaccharides, glucose, etc.) may be cited.

In the following step “(9) consultation”, the subject person gets advice about the recovery care to be carried out at his/her own home before the next treatment. An important point in the above-described recovery care resides in that the skin should be rested 2 or 3 days after the beauty treatment performed by the skilled persons and then subjected to the careful recovery treatment. That is to say, the skin, which has been relatively strongly stimulated by the above-described beauty treatment, is in preparation for improvement. During this period, it is recommended not to stimulate the skin any more. When the skin gets in the course of improvement, then active components are carefully supplied so that the sin conditions can upgrade.

The entire body care course comprising a series of treatments has completed by the above-described (1) to (9). After coming home, it is desirable that the client carries out there covery care given in the frame 2 in FIG. 1 by himself/herself depending on the above-described advice.

The above-described recovery care is carried out in 2 steps including “(a) skin rest” and “(b) recovery treatment” depending on the above-described advice.

In the step “(a) skin rest”, the skin is allowed to rest for 2 or 3 days after having the above-described beauty treatment and coming home so as to minimize stimulations on the skin. For example, massages or the like should be avoided and a merely less stimulating skin care treatment such as moisturizing with a lotion mask may be carried out.

In the following step “(b) recovery treatment”, the client himself/herself actively performs some treatments such as massaging or using a mask that is rich in active components. Further improved effects can be established by combining the intake of a supplement before or after the recovery treatment, bathing before or during the recovery treatment or napping or sleeping after the recovery treatment.

According to the cosmetic method comprising the entire body care course as described above, the 3 types of medicinal components having been orally taken are effectively absorbed into the entire body owing to the combination thereof with the bathing treatment, the beauty treatment for the full body, the napping treatment, etc. As a result, excellent cosmetic effects that cannot be obtained by the existing methods can be established. According to the cosmetic method comprising the combination of the above-described entire body care course with the recovery care at home, sufficient rest and new stimulation are provided to the skin in the course of the recovery of the skin from the stimulations given by the beauty treatment and so on in the entire body care course. As a result, the skin conditions can upgrade and still improved cosmetic effects can be obtained.

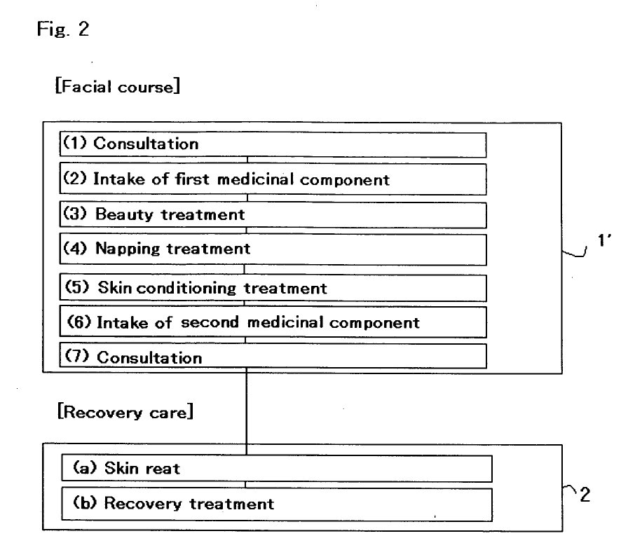

The course given in the frame 1′ in FIG. 2 and named “facial course” is to be prepared together with the entire body care course as described above. This course has the following 7steps: (1) consultation; (2) intake of a first medicinal component; (3) a beauty treatment; (4) a napping treatment; (5) a skin conditioning treatment; (6) intake of a second medicinal component; and (7) consultation.

The first step “(1) consultation” in the above-described facial course is carried out as in the first consultation step in the above-described entire body care course.

In the following step “(2) intake of a first medicinal component”, use is made of a supplement containing a medicinal component suitable for the subject person's conditions which is prepared based on the treatment plan determined in “(1) consultation” as described above. The above-described supplement may be appropriately selected from among those listed in the above Table 1 and blended. In this step, it is usually appropriate to take a supplement in the form of a concentrated drink that contains a component having skin care effect. As an example of the formulation, a concentrated drink containing a mixture of vitamin C, vitamin B₁₂, vitamin E, vitamin B family, beta-carotene and amino acids at an appropriate ratio may be cited.

In the following step “(3) beauty treatment”, the following operations are performed on the subject person. Namely, the subject person has hand massages on the head with the use of a massage oil. The face and the part from the neck to the breast (the decollete) are subjected to a series of operations including: cleansing→ hand massaging with the use of a massage agent→ impartment of an active ingredient by masking and impartment of a cosmetic lotion, an emulsion and a cosmetic essence suitable for the skin conditions of the subject person. Also, a powder massage using a body powder is performed. These operations for respective parts may be performed in an optional order. Similar to the hand massages in the entire body care course, hand massages in this step are performed mainly in 3 steps including physically stimulating operations for promoting blood circulation such as rubbing, kneading and tapping, balancing operations such as pushing reflex points and finger pressure, and operations for maintaining improved blood circulation such as stroking the skin and heating. It is also possible to appropriately combine these operations with blood circulation-promoting treatments such as wrapping and packing.

In the following step “(4) napping treatment”, the subject person is allowed to have a nap. In the following step “(5) skin conditioning treatment”, a skin conditioning treatment is carried out on the subject person. The constitutions and effects of these steps are similar to those in the entire body care course.

In the following step “(6) intake of a second medicinal component”, supplement containing a medicinal component suitable for the subject person's conditions is prepared and orally administered to the subject person. The above-described supplement may be appropriately selected from among those listed in the above Table 1 and blended. In this step, it is usually appropriate to select a supplement having an awakening effect and an energy-supplying effect. As an example of the formulation, a candy containing a mixture of glucose, niacin vitamins such as niacin, amino acids, hydrocarbonates (monosaccharides, glucose, etc.) may be cited.

Finally, the step “(7) consultation” is performed as in the entire body care course and thus the facial course is completed.

After coming home, it is desirable that in the home, the client carries out the recovery care including “(a) skin rest” and “(b) recovery treatment” by himself/herself depending on the above-described advice, similar to the client having the above-described entire body care course. The above-described recovery care is the same as the recovery care to be carried out following the entire body care course and, therefore, illustration thereof is omitted.

According to the cosmetic method comprising the facial course as described above, the 2 types of medicinal components having been orally taken are effectively absorbed into the body owing to the combination thereof with the beauty treatment mainly for the face, etc. As a result, excellent cosmetic effects that cannot be obtained by the existing methods can be established. According to the cosmetic method comprising the combination of the above-described facial course with the recovery care at home, sufficient rest and new stimulation are provided to the skin in the course of the recovery of the skin from the stimulations given by the beauty treatment and so on. As a result, the skin conditions can upgrade and still improved cosmetic effects can be obtained.

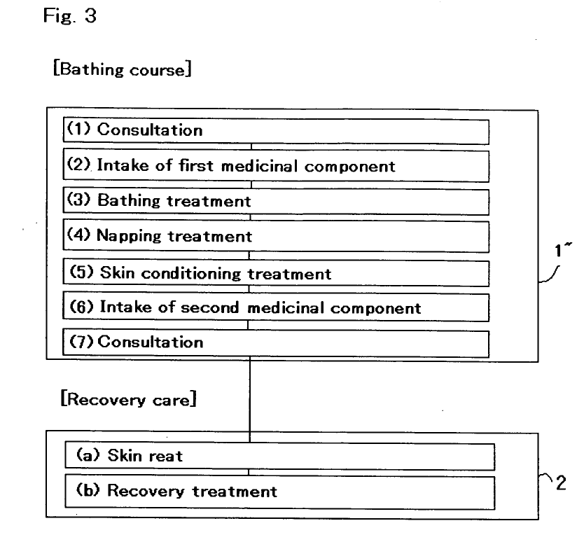

The course given in the frame 1″ in FIG. 3 and named “bathing course” is to be prepared together with the entire body care course and the facial course as described above. This course has the following 7 steps: (1) consultation; (2) intake of a first medicinal component; (3) a bathing treatment; (4) a napping treatment; (5) a skin conditioning treatment; (6) intake of a second medicinal component; and (7) consultation.

Similar to the other courses, the first step “(1) consultation” in the above-described bathing course is carried out. In the following step “(2) intake of a first medicinal component”, a supplement is administered to a subject person. The above-described supplement may be the same as the first medicinal component in the entire body care course as described above.

Subsequently, the steps “(3) a bathing treatment”, “(4) a napping treatment” and “(5) a skin conditioning treatment” are carried out as in the above-described entire body care course.

In the following step “(6) intake of a second medicinal component”, a concentrated drink comprising an appropriate mixture of supplements having a skin care effect as the first medicinal component in the above-described entire body care course is administered to the subject person.

Finally, the step “(7) consultation” is performed as in the entire body care course and the facial course as described above and thus the bathing course is completed.

After coming home, it is desirable that in the home, the client carries out the recovery care including “(a) skin rest” and “(b) recovery treatment” by himself/herself depending on the above-described advice, similar to the client having the above-described entire body care course or the facial course.

According to the cosmetic method comprising the bathing course as described above, the 2 types of medicinal components having been orally taken are effectively absorbed into the body owing to the combination thereof with the bathing treatment, etc. As a result, excellent cosmetic effects that cannot be obtained by the existing methods can be established. According to the cosmetic method comprising the combination of the above-described bathing course with the recovery care at home, sufficient rest and new stimulation are provided to the skin in the course of the recovery of the skin from the stimulations given by the beauty treatment and so on. As a result, the skin conditions can upgrade and still improved cosmetic effects can be obtained.

In each of the above-described cosmetic methods, therefore, the medicinal components having been orally taken are effectively absorbed into the body owing to the combination thereof with various treatments. As a result, excellent cosmetic effects can be established. Therefore, a client can select the most suitable course for him/her from among the above-described 3 courses depending on the skin conditions, margin for time and so on. By performing the recovery care at his/her own home according to the advice, moreover, still improved cosmetic effects can be obtained.

Although the 3 courses, i.e., the entire body care course, the facial course and the bathing course are prepared in the above example, it is not always necessary to prepare all of these 3courses. Namely, at least one of these courses is to be prepared. Concerning the contents of the courses, the constitution of each course are not specifically restricted so long as it involves combination of a step of taking a medicinal component with at least one of a beauty treatment (either for the entire body or for the face, etc.), a beauty treatment and a napping treatment.

It is not always necessary that a subject person performs the recovery care at his/her own home after the treatments have been carried out by, for example, an esthetician in an esthetic salon as in the above-described cases. However, it is favorable for achieving improved cosmetic effects to perform the recovery care as discussed above.

Supplements for taking medicinal components are not restricted to those described in the above cases but use can be made of various supplements. Furthermore, it is also possible to employ sweet-smelling components or music during optional treatments or in the wait time for the next treatment for the body and mind relaxation, though these factors are not orally taken. Needless to say, it is also possible to appropriately impregnate nice smell on a massage agent, a cosmetic liquid preparation, acosmeticlotion, etc. to be used in the treatments.

Moreover, the contents of the consultation and the hand massage operations, etc. in the beauty treatment are not restricted to those described in the above cases but optional selection may be made therefor.

1. A cosmetic method comprising making a subject person to take a medicinal component and then carrying out at least one of a bathing treatment, a beauty treatment and a napping treatment.

2. A cosmetic method as claimed in claim 1 comprising making a subject person to take a medicinal component and then carrying out a bathing treatment followed by a beauty treatment.

3. A cosmetic method comprising making a subject person to take a first medicinal component, then carrying out a bathing treatment, making the subject person to take a second medicinal component and then carrying out a beauty treatment.

4. A cosmetic method comprising making a subject person to take a first medicinal component, then carrying out a bathing treatment, making the subject person to take a second medicinal component, then carrying out a beauty treatment followed by a napping treatment and then making the subject person to take a third medicinal component.

5. A cosmetic method comprising carrying out a cosmetic method as claimed in claim 1 and then making a subject person to carry out a recovery care comprising a skin rest treatment combined with a skin recovery treatment in the home, etc.

6. A cosmetic method comprising carrying out a cosmetic method as claimed in claim 2 and then making a subject person to carry out a recovery care comprising a skin rest treatment combined with a skin recovery treatment in the home, etc.

7. A cosmetic method comprising carrying out a cosmetic method as claimed in claim 3 and then making a subject person to carry out a recovery care comprising a skin rest treatment combined with a skin recovery treatment in the home, etc.

8. A cosmetic method comprising carrying out a cosmetic method as claimed in claim 4 and then making a subject person to carry out a recovery care comprising a skin rest treatment combined with a skin recovery treatment in the home, etc.

9. A method of promoting cosmetic effects in the body of a subject person, comprising:
selecting a medical component or supplement having cosmetic effects;
administrating an effective amount of the medical component or supplement to the subject person, thereby allowing the medical component or supplement to be absorbed in blood of the subject person; and
at, near, or during the time an increase of concentration of the medical component or supplement in blood occurs, increasing blood circulation in the body by applying external treatment to the subject person, thereby promoting absorption of the medical component or supplement in the body.

10. The method as claimed in claim 9, further comprising discontinuing the external treatment to allow the subject person to rest without significantly decreasing the concentration of the medical component or supplement in blood; and administrating an effective amount of a second medical component or supplement having cosmetic effects to the subject person.

11. The method as claimed in claim 9, wherein the external treatment is at least one of a bathing treatment or a beauty treatment.

12. The method as claimed in claim 10, wherein the resting is carried out by a napping treatment.

13. The method as claimed in claim 9, wherein the medical component or supplement is orally administrated.

14. The method as claimed in claim 9, wherein the external treatment in the step of increasing blood circulation is conducted on a skin, thereby increasing blood circulation in the skin.
